(19) United States
(12) Patent Application Publication
(10) Pub. No.: US 2009/0220426 A1
(43) Pub. Date: Sep. 3, 2009

(54) Biodegradable Inverted-Opal Structure, Method for Manufacturing and Using the Same, and Medical Implant Comprising the Biodegradable Inverted-Opal Structure

(75) Inventors: Musashi Fujishima, Osaka (JP); Kumao Uchida, Shiga (JP)

Correspondence Address: LAWSON & WEITZEN, LLP, 88 BLACK FALCON AVE, SUITE 345, BOSTON, MA, 02210, US

(73) Assignee: KINKI UNIVERSITY, Higashi-Osaka-shi (JP)

(21) Appl. No.: 12/223,344

(22) PCT Filed: Jan. 18, 2007
(86) PCT No.: PCT/JP2007/050722

§ 371 Date: Dec. 24, 2008

(30) Foreign Application Priority Data
Jan. 30, 2006 (JP) 2006-021504

(51) Int. Cl. A61K 9/00; B05D 3/00; B05D 3/10; A61P 43/00; A61K 47/34

(52) U.S. Cl. 424/9.2; 424/426; 427/2.24; 514/772.3

(57) ABSTRACT

(Problems) The object of the present invention is to provide an inverted-opal structure which is excellent in biodegradability, biocompatibility, and pH responsiveness, has specific light reflection property due to three-dimensionally-ordered-pores formed therein, is capable of releasing a drug autonomously and intermittently by responding rapidly to pH change, and is capable of measuring the drug-release associated with its biodegradation by an optical means rapidly in a simple and easy way; a method for manufacturing the inverted-opal structure; a medical implant comprising the inverted-opal structure; a method for enlarging the pore diameter; and a method for measuring the release-amount of a drug held in the inverted-opal structure.

(Means for Solving Problems) The present invention provides a biodegradable inverted-opal structure comprising an aliphatic polyester; and a method for manufacturing a biodegradable inverted-opal structure, comprising the steps of: (1) producing a colloidal crystal from a silica particle or a polystyrene particle; (2) immersing the colloidal crystal in a solution including a monomer from which the aliphatic polyester is formed; (3) thermally-polymerizing the monomer under a pressurized condition in order to obtain a composition of the colloidal crystal coated with the aliphatic polyester; and (4) removing the silica particle from said composition by etching, or removing the polystyrene particle from said composition by eluting the polystyrene particle with an organic solvent in order to obtain the biodegradable inverted-opal structure.

### FIELD OF THE INVENTION

This invention relates to a biodegradable inverted-opal structure, methods for manufacturing and using the same, and a medical implant comprising the biodegradable inverted-opal structure. Specifically, the biodegradable inverted-opal structure of the present invention is preferably used for medical field because it has biodegradability, biocompatibility, light reflection property and pH responsiveness.

### DESCRIPTION OF THE RELATED ART

In medical and pharmaceutical fields, a system to release an effective amount of agent for a required time in a specific part of biomedical tissue while exhibiting the effect of the agent sufficiently has been highly demanded. Such a system enables to inhibit side-effects caused by the agent. Thus, an implant comprising a carrier capable of holding an agent has been widely invented.

For example, Patent document 1 discloses a stimulus-responsive porous polymer gel which changes its structural color by responding the changes of temperature, sugar concentration and ion concentration. Further, various measuring reagents comprising the above-mentioned polymer gel is disclosed in Patent document 1. According to Patent document 1, although the stimulus response speed of the disclosed invention may be fast, it requires an organic solvent and a polymerization initiator in order to synthesize the polymer gel. Thus, it is concerned that the biological toxicity, which is caused by unreacted reagents and residues derived from the solvent and polymerization initiator, exists in the polymer gel, and therefore the polymer gel is not appropriately used as a medical implant for biomedical tissue.

In Patent document 2, a three-dimensionally periodic structure, which is a non-inverted-opal-structured-form, comprising a polylactic acid, and a manufacturing method thereof are disclosed. The process for manufacturing a porous substrate to be used as a template, which is disclosed in Patent document 2, requires certain conditions, and the conditions are difficult to be adjusted. Further, the substrate can not be applied to a low fluidity polymer or a polymer gel due to their poor penetrability. Further, the structure obtained from Patent document 2 has an internal space which is relatively small, so that the amount of holding drug is limited. In addition, this structure comprises the polylactic acid which is electrostatically neutral, and therefore has low ability of responding to the physicochemical environmental-changes. Although, this structure is capable of releasing a drug continuously due to the natural decomposition, the structure can not release a drug intermittently and rapidly according to a mechanical response to pH change in a biomedical tissue. Also, this structure does not have enough compatibility with hydrophilic environments such as biomedical tissue and can not firmly hold a hydrophilic drug.

Patent document 3 discloses that a medical implant comprising a biodegradable polymer is biodegraded in vivo, so that a holding-drug is continuously released to the location of a lesion. However, specifically when a drug having strong side-effects is used, it is desirable to release the holding-drug intermittently rather than continuously by autonomously responding to the physicochemical environmental-changes which particularly occurs in the location of a lesion. Also, the medical implant has a problem that its drug-release amount is only observed indirectly by measuring the changes in size and shape of the implant during the biodegradation with large equipments such as X-ray CT and MRI.

Patent document 4 discloses a mesh structure which is considered to have two-dimensionally-arranged-voids. Such a structure has the problem that its selective light reflection property and mechanical responsiveness are low. Further, after the structure disclosed in Patent document 4 is buried into a biomedical tissue, it is necessary to use large equipments such as X-ray CT and MRI in order to measure the residual volume during the biodegradation. Such large equipments are physically-taxing to the treated patients.

Patent document 5 discloses a biodegradable polymer comprising a copolymer of polylactic acid and polyglycolic acid. This copolymer is a straight-chain polymer and has a non-porous structure, and therefore it shows low mechanical responsiveness. Further, the invention disclosed in Patent document 5 needs troublesome steps in order to completely remove an organic solvent which is used for its synthesis, and therefore the manufacturing efficiency is low.

Patent document 6 discloses an inverted-opal structure comprising a composition including a sulfide series compound such as episulfide compound as an essential component. The inverted-opal structure is a composition having high refractive index, and it is not appropriate for medical use. The structure is manufactured with the aim of being applied to optical devices such as optical filter, optical waveguide and laser cavity. Thus, the structure disclosed in Patent document 6 does not have sufficient biodegradability and biocompatibility (ex. non-stimulus property, low drug toxicity caused by the degraded products) which are required for use in a biomedical tissue.

The structure disclosed in Non Patent document 1 is a non-porous body, and therefore its refractive index is uniformity. Thus, the non-porous body does not show reflection property. Further, the non-porous body is not expected to have a sufficient mechanical response speed (ex. swelling and contraction) against external stimuli such as pH.

Thus, the following properties are desired, however, a structure having such properties has not been invented under the present circumstances.

Excelling in biodegradability, biocompatibility, and pH responsiveness

Having specific light reflection property due to the three-dimensionally-ordered-pores

Being capable of releasing a drug autonomously and intermittently by responding rapidly to pH change

Being capable of observing the drug-release resulting from the biodegradation by an optical means rapidly in a simple and easy way

[Patent document 1] Japanese patent publication 2004-27195
[Patent document 2] International patent publication
[Patent document 3] Japanese patent publication 10-505587
[Patent document 4] Japanese patent publication
[Patent document 5] Japanese patent publication
[Patent document 6] Japanese patent publication 2004-17044
[Non Patent document 1] The society of polymer science preceding manuscript, Vol. 50, No. 4, P835, 2001

### DISCLOSURE OF THE INVENTION

### Problems to be Solved by the Invention

The present invention is invented in order to solve the problems in the prior arts. The present invention aims at providing a biodegradable inverted-opal structure excelling in biodegradability, biocompatibility and pH responsiveness, and having specific light reflection property due to the three-dimensionally-ordered-pores, and a method for manufacturing the same.

Another aim of the present invention is to provide a biodegradable inverted-opal structure and a medical implant capable of releasing a drug autonomously and intermittently by responding rapidly to pH change and observing the drug-release resulting from the biodegradation by an optical means rapidly in a simple and easy way.

Another aim of the present invention is to provide methods for enlarging a pore diameter of the biodegradable inverted-opal structure and for measuring a drug-release amount of the drug which is held in the biodegradable inverted-opal structure.

### Means for Solving the Problems

The inventors found that a medical implant excelling in biodegradability, biocompatibility and pH responsiveness and having extremely high availability is manufactured by using an inverted-opal structure having three-dimensionally-ordered-pores, and thereby developed this invention.

The present invention according to claim 1 relates to a biodegradable inverted-opal structure comprising an aliphatic polyester.

The present invention according to claim 2 relates to the biodegradable inverted-opal structure according to claim 1, wherein said inverted-opal structure has three-dimensionally-ordered-pores, and said pore selectively reflects light in visible and near-infrared regions.

The present invention according to claim 3 relates to the biodegradable inverted-opal structure according to claim 2, wherein said light in visible and near-infrared regions has a wavelength of 600 to 1100 nm.

The present invention according to claim 4 relates to the biodegradable inverted-opal structure according to claim 2 or 3, wherein said pore has a diameter of 10 to 1000 nm.

The present invention according to claim 5 relates to the biodegradable inverted-opal structure according to any of claims 1 to 4, wherein said aliphatic polyester is formed by ester-bonding between monomers selected from the group consisting of polyhydric carboxylic acid, polyhydric alcohol, hydroxycarboxylic acid and lactone-group.

The present invention according to claim 6 relates to the biodegradable inverted-opal structure according to claim 5, wherein said aliphatic polyester comprises said monomers in a composition rate ranged from 0.001 to 1000% by weight respectively.

The present invention according to claim 7 relates to the biodegradable inverted-opal structure according to any of claims 1 to 6, wherein said aliphatic polyester is a polylactic acid.

The present invention according to claim 8 relates to the biodegradable inverted-opal structure according to any of claims 1 to 7, wherein said inverted-opal structure has a pH responsiveness.

The present invention according to claim 9 relates to a medical implant comprising the biodegradable inverted-opal structure according to any of claims 1 to 8.

The present invention according to claim 10 relates to a composition of a colloidal crystal coated with an aliphatic polyester manufactured by a method comprising the steps of: (1) producing a colloidal crystal from a silica particle or a polystyrene particle; (2) immersing the colloidal crystal in a solution including a monomer from which the aliphatic polyester is formed; and (3) thermally-polymerizing the monomer under a pressurized condition in order to obtain a composition of the colloidal crystal coated with the aliphatic polyester.

The present invention according to claim 11 relates to the composition of the colloidal crystal coated with the aliphatic polyester according to claim 10, wherein said silica particle or polystyrene particle has a weight fraction of 0.01-90% by weight.

The present invention according to claim 12 relates to a method for manufacturing a biodegradable inverted-opal structure, comprising the steps of: (1) producing a colloidal crystal from a silica particle or a polystyrene particle; (2) immersing the colloidal crystal in a solution including a monomer from which the aliphatic polyester is formed; (3) thermally-polymerizing the monomer under a pressurized condition in order to obtain a composition of the colloidal crystal coated with the aliphatic polyester; and (4) removing the silica particle from said composition by etching, or removing the polystyrene particle from said composition by eluting the polystyrene particle with an organic solvent in order to obtain the biodegradable inverted-opal structure.

The present invention according to claim 13 relates to a method for using a biodegradable inverted-opal structure comprising an aliphatic polyester, comprising a step of releasing a drug from the biodegradable inverted-opal structure in vivo by biodegrading and/or responding to pH after holding said drug in the biodegradable inverted-opal structure.

The present invention according to claim 14 relates to a method for measuring a drug-release amount in vivo from a biodegradable inverted-opal structure comprising an aliphatic polyester, comprising the steps of: (a) releasing a drug from the biodegradable inverted-opal structure in vivo by biodegrading and/or responding to pH after holding said drug in the biodegradable inverted-opal structure; and (b) entering light in visible or near-infrared region into said biodegradable inverted-opal structure, and measuring the change of wavelength and strength of the reflected light.

The present invention according to claim 15 relates to a method for measuring a drug-release amount in vivo from a biodegradable inverted-opal structure comprising an aliphatic polyester according to claim 14, further comprising the steps of: (i) releasing a pseudo drug from the biodegradable inverted-opal structure in vivo by biodegrading and/or responding to pH after holding said drug in the biodegradable inverted-opal structure; and (ii) entering light in visible or near-infrared region into said biodegradable inverted-opal structure, measuring (A) change of wavelength and strength of the reflected light, measuring (B) drug-release amount of said pseudo drug by a quantitative analysis of visible absorption spectrum, and correlating (A) with (B).

The present invention according to claim 16 relates to a method for enlarging a pore diameter of a biodegradable inverted-opal structure comprising an aliphatic polyester, comprising a step of hydrolyzing the inner wall of the pore of the biodegradable inverted-opal structure.

### EFFECT OF THE INVENTION

The biodegradable inverted-opal structure of the present invention excels in biodegradability, biocompatibility and pH responsiveness. The biodegradable inverted-opal structure of the present invention has specific light reflection property due to the three-dimensionally-ordered-pores.

The biodegradable inverted-opal structure of the present invention is excellent in pH responsiveness, and therefore it enables to release a drug by responding autonomously and rapidly to a cancer tissue having low pH conditions. In addition, the biodegradable inverted-opal structure of the present invention has specific light reflection property. Thus, the biodegradable inverted-opal structure of the present invention has the property of selectively reflecting light in visible and near-infrared regions having high tissue penetration and low-barrier, and therefore it enables to measure the drug-release amount with an optical means.

The medical implant of the present invention comprises the biodegradable inverted-opal structure having the above-mentioned effects, and therefore it is preferably used in a medical field and applied to a localized chemical treatment of cancer or the like.

The method for manufacturing the biodegradable inverted-opal structure of the present invention can produce the biodegradable inverted-opal structure having the above-mentioned effects in a simple and easy way.

The method for measuring the drug-release amount from the biodegradable inverted-opal structure enables to reduce burdens of patients and measuring the drug-release amount easily. Further, the value of the drug-release amount is accurately measured by using the above-mentioned measuring, so that the method is preferably used in a medical field.

According to the method for enlarging a pore diameter of the biodegradable inverted-opal structure of the present invention, the pore diameter is easily enlarged by regulating pH, and therefore the holding-drug-release may be regulated.

### DESCRIPTION OF THE PREFERRED EMBODIMENTS

Hereinafter, a biodegradable inverted-opal structure of the present invention will be explained.

The biodegradable inverted-opal structure having three-dimensionally-ordered-pores of the present invention has a structure in which pores having a diameter of almost the same as light wavelength are ordered three-dimensionally and periodically. Such an opal structure is known that it selectively reflects the light of specific wavelength and has a structural color such as shown in natural opals. In addition, the structure is characterized in having a large specific surface area derived from the porous structure, and therefore it has 3 or 4 digit higher mechanical response speed against external stimuli than a non-porous polymer.

The biodegradable inverted-opal structure of the present invention is characterized in comprising an aliphatic polyester.

The reason for using the aliphatic polyester is that the aliphatic polyester is excellent in biodegradability and biocompatibility and is capable of responding to pH. Another reason is that the aliphatic polyester can be synthesized by a thermal polymerization reaction in an aqueous system, and therefore an organic solvent or a polymerization initiator is not necessarily used. This avoids biological toxicity caused by the residues.

The aliphatic polyester according to the present invention is preferably synthesized by using one or more monomers selected from the group consisting of polyhydric carboxylic acid, polyhydric alcohol, hydroxycarboxylic acid and lactone-group. The aliphatic polyester is easily synthesized by a condensation-polymerization reaction in the aqueous system without using a polymerization initiator. However, the aliphatic polyester of the present invention may be synthesized by using an organic solvent and a polymerization initiator. As for combinations of the above-monomers, polyhydric carboxylic acid and polyhydric alcohol, polyhydric carboxylic acid and hydroxycarboxylic acid, polyhydric alcohol and hydroxycarboxylic acid are used. In addition, the aliphatic polyester may be obtained by a condensation-polymerization reaction between hydroxycarboxylic acids. A composition rate of these combinations is optionally decided, but the range from 0.001 to 1000% by weight respectively is preferably used, and more preferably the range from 0.1 to 90% by weight is used. The reason is that when the composition rate is in the range from 0.001 to 1000% by weight, carboxyl group and hydroxyl group which are not involved in the ester-bond exist, and therefore excellent biodegradability and pH responsiveness are achieved.

As for the polyhydric carboxylic acid according to the present invention, a compound structurally having two or more carboxyl groups is preferably used. The examples are shown as below.

Citric acid, malic acid, acidum tartaricum, phthalic anhydride, terephthalic acid, maleic acid, fumaric acid, succinic acid, adipic acid, malonic acid, oxalic acid, pimelic acid, glutaric acid, suberic acid, azelaic acid, sebacic acid, undecanedoic acid, dodecanedioic acid, butyric acid, valeric acid, aconitic acid, glutamic acid, asparagine acid, acetoxy succinic acid, isocamphoric acid, itaconic acid, ethylmalonic acid, oxaloacetic acid, oxydiacetic acid, carboxy oxanilic acid, citraconic acid, citramalic acid, dimethyl succinic acid, dimethyl malonic acid, tetramethyl succinic acid, tridecanedioic acid, methylmalonic acid, methyl succinic acid, mesaconic acid, hexadienedioic acid, 1,2,3-propanetricarboxylic acid, crotonic acid, citraconic acid and oxoheptane acid are exampled.

As for the polyhydric alcohol according to the present invention, a compound structurally having two or more hydroxyl groups is preferably used. The examples are shown as below.

Pentaerythritol, dipentaerythritol, tripentaerythritol, ethylene glycol, diethylene glycol, triethylene glycol, tetraethylene glycol, propylene glycol, dipropylene glycol, butanediol, pentanediol, neopentyl glycol, glycerin, 1,6-hexanediol, 1,9-nonanediol, monopalmitin, monostearin, monoacetin, monoolein, 3-methoxy-2,3-butanediol, hexanediol, 2-butyne-1,4-diol and 2-methylene-1,2-propanediol are exampled.

As for the hydroxycarboxylic acid according to the present invention, a compound structurally having one or more hydroxyl group and one or more carboxyl group is preferably used. The examples are shown as below.

Lactic acid, glycolic acid, mandelic acid, isovanillic acid, glyceric acid, glutaconic acid, serine, hydracrylic acid, 10-hydroxyoctadecanoic acid, hydroxyglutaric acid, 2-hydroxy-2-methylpropionic acid, hydroxybutyric acid, pinacol, ricinelaidic acid, o-lactyl lactic acid, and tetrahydroxybutyric acid are exampled.

Further, as for the monomer according to the present invention, lactone-group having a cyclic structure may be used. The examples are shown as below.

β-propiolactone, β-butyrolactone, pivalolactone, β-benzylmalolactonate, γ-butyrolactone, γ-valerolactone, σ-valerolactone, ε-caprolactone, lactone, pantolactone, paraconic acid, terebic acid, diketene, equilin, glycolide, lactide, and malidebenzylester are exampled.

Among the above-monomers, the preferable combination for the aliphatic polyester according to the present invention is not limited to, but the combinations of citric acid and pentanediol, citric acid and pentaerythritol, citric acid and lactic acid, citric acid and glycolic acid, malic acid and lactic acid, and malic acid and glycolic acid may be used.

Alternatively, as for aliphatic polyester according to the present invention, polylactic acid is preferably used. The polylactic acid may be used in any of two enantiomers (i.e., D-type and L-type) and DL-type comprising the D-type and L-type. The polylactic acid whose molecular weight is in the range of 1,000 to 10,000,000 is used, and preferably in the range of more than 10,000 may be used. The polylactic acid having such a molecular weight has high regularity in the three-dimensionally-pore-structure of the inverted-opal structure, excels in its mechanical strength and has low biodegradable speed, and therefore it is preferably used.

The biodegradable inverted-opal structure according to the present invention structurally has carboxyl group which is derived from polyhydric carboxylic acid or hydroxycarboxylic acid. The carboxyl group concentration in the aliphatic polyester may be determined by the monomer types and the composition rate during the synthesis. This allows the hydrophilia to be regulated. This results in having excellent compatibility for a biomedical tissue and regulatable biodegradability. Further, because the biodegradable inverted-opal structure according to the present invention has a mechanical contraction and swelling property caused by the proton addition and dissociation occurring in the carboxyl group, it enables to autonomously respond to the low-pH conditions such as a cancer tissue.

Next, the shape of the biodegradable inverted-opal structure according to the present invention will be explained.

The biodegradable inverted-opal structure according to the present invention has three-dimensionally-ordered-pores therein. The structure is formed by a template consisting of a colloidal crystal which has a three-dimensionally-ordered-structure.

The pore has a diameter of preferably 10 to 1000 nm, and more preferably 200 to 600 nm. Because the biodegradable inverted-opal structure of the present invention has such a pore, it is able to selectively reflect the light of specific wavelength. The wavelength of reflected light is changed depending on the incidence angle of light, the pore diameter, the volume fraction of materials existing in the pore to the inverted-opal structure and the refractive index, based on Bragg's law and Snell's low. As for the light of specific wavelength, for example, visible and near-infrared light having the wavelength of 600 to 1100 nm is used.

The biodegradable inverted-opal structure of the present invention has high tissue penetration. Further, because the structure has a large specific surface area compared to a non-porous polymer, it excels in responding rapidly to pH change and selectively reflecting the light in visible and near-infrared regions. Thus, the biodegradable inverted-opal structure of the present invention is preferably used as a medical implant by being buried in a biomedical tissue. Specifically, the implant is used as an implant for holding platinum-containing drug, antibacterial agent, hormonal agent, DNA agent or the like. Further, the implant is used as an implant for a localized chemical treatment of cancers such as brain tumor by holding alkylating agents such as ACNU and BCNU.

Next, a method for manufacturing the biodegradable inverted-opal structure of the present invention will be explained.

The method for manufacturing the biodegradable inverted-opal structure of the present invention is characterized in comprising the following steps (1) to (4).

(1) producing a colloidal crystal from a silica particle or a polystyrene particle;
(2) immersing the colloidal crystal in a solution including a monomer from which the aliphatic polyester is formed;
(3) thermally-polymerizing the monomer under a pressurized condition in order to obtain a composition of the colloidal crystal coated with the aliphatic polyester; and
(4) removing the silica particle from said composition by etching, or removing the polystyrene particle from said composition by eluting the polystyrene particle with an organic solvent in order to obtain the biodegradable inverted-opal structure.

In the step (1), a colloidal crystal is produced from a silica particle or a polystyrene particle.

The biodegradable inverted-opal structure of the present invention is preferably manufactured by a replica method using the colloidal crystal as the template. As a simple preparation method of the colloidal crystal, a gravity sedimentation method is exampled. This method takes advantage of a self-accumulation property of silica sol. This property results from a capillary-force in the transverse direction between silica particles when the solvent is gradually evaporated from the colloidal suspension which is delivered by drops onto the substrate. In this method, only a low-crystalline colloidal crystal may be obtained. However, a colloidal crystalline film having a relatively large area may be prepared by covering the surface of solvent with a nonvolatile material. Besides this method, electrochemical self-accumulation method and hydrodynamic accumulation method may be also used in order to prepare a colloidal crystal having a high-three-dimensional-regularity.

In the present invention, as for the colloidal particle having uniform particle size, silica particle and polystyrene particle may be preferably used. For example, such particles having a diameter of 3 nm to 90 nm are sold at a relatively low price. Though depending on its synthesis conditions, the colloidal crystal, which is used as the template, normally forms a cubic closest-packed structure, and the lattice constant may be regulated according to the colloidal particle diameter. In order to selectively reflect the light ranged from visible to near-infrared regions, the colloidal particle diameter is not limited to, but preferably 200 to 600 nm, and more preferably 300 to 500 nm. The appearance of the colloidal crystal according to the present invention is shown in FIG. 1(1).

In the step (2), the colloidal crystal obtained from the step (1) is immersed in a solution including a monomer from which the aliphatic polyester is formed. When the colloidal crystal has a face-centered cubic structure, 74% of the volume fraction is occupied by the colloidal crystal, and 26% of the volume fraction, which is void, is occupied by the monomer solution. In addition, when the colloidal crystal has a different structure from the above-structure and when the colloidal crystal is prepared by a mixed solution of colloidal suspension and monomer solution, their volume fraction is not limited to the above-mentioned volume fraction.

In the step (3), a composition of the colloidal crystal coated with the aliphatic polyester is obtained by thermally-polymerizing the monomer under a pressurized condition.

This thermal-polymerization may be carried out under pressure with moisture vapor or the like according to the present invention. This prevents from generation of air bubbles in the polymer which is caused by the boiling of the monomer solution associated with the polymerization under high temperature. By this way, the aliphatic polyester having no air bubbles therein may be obtained. In the present invention, for example, a pressure bottle is preferably used.

The temperature of the above thermal-polymerization may be preferably 50 to 150° C., and more preferably 80 to 130° C. In the condensation polymerization reaction, multiple ester-bonds are formed between monomers, and the polymer gel having a straight-chain polymer or a three-dimensional mesh polymer is obtained.

Also, the air bubbles generation in the aliphatic polyester is controlled by adjusting both of the temperature and the pressure power.

In the composition of the colloidal crystal coated with the aliphatic polyester, the silica particle or polystyrene particle has a weight fraction of preferably 0.01-90% by weight, and more preferably 0.1-50% by weight. The reason is that when the ratio of silica particle or polystyrene particle is in the range of 0.01-90% by weight, the colloidal crystal excels in the three-dimensional periodicity. The composition of the colloidal crystal coated with the aliphatic polyester obtained from the step (3) is shown in FIG. 1(2).

Next, in the step (4), the silica particle used as the template which is internally located in the colloidal crystal coated with the aliphatic polyester is removed by etching with a solution such as hydrogen fluoride. Alternatively, in the step (4), polystyrene particle is removed by being eluted with an organic solvent. With either the treatment, the biodegradable inverted-opal structure is obtained.

As for the organic solvent, for example, toluene is used.

The biodegradable inverted-opal structure obtained from the step (4) is shown in FIG. 1(3).

The obtained biodegradable inverted-opal structure has preferably a thin-filmy shape. However, various shapes of the biodegradable inverted-opal structure, such as acicular, wafer and pellet form, may be obtained by using the silica particle or polystyrene particle having an appropriate diameter or using a suitable shaped container.

The pore diameter of the biodegradable inverted-opal structure obtained from the step (4) depends on the diameter of the colloidal crystal used as the template. However, the pore diameter may be changed after the biodegradable inverted-opal structure is manufactured. For example, the pore diameter may be enlarged by hydrolyzing the inner wall of the pore with a buffer solution or an enzyme, or immersing the structure in the solution whose pH is regulated to a given value. Further, the pore diameter may be reduced by immersing the structure in the polymer solution diluted to a proper concentration and then carrying out the thermal polymerization.

Next, a method for using the biodegradable inverted-opal structure of the present invention will be explained.

The biodegradable inverted-opal structure of the present invention holds a drug in the pore of the biodegradable inverted-opal structure. The structure holding the drug is buried in a biomedical tissue and releases a drug by biodegrading and/or responding to pH.

As for the drug, it is not limited to, but a drug having a low solvent solubility or a drug being easily degraded in vivo is preferably held in the biodegradable inverted-opal structure of the present invention because the structure is in a solid state. For the detail, alkylating agents such as ACNU and BCNU or the like, platinum-containing drug, antibacterial agent and hormonal agent are exampled, and further, DNA agent or the like may be held in the structure. In addition, because the hydrophilic conditions of the structure are able to be adjusted, it is also appropriate to hold a highly-hydrophilic drug.

In order to hold the drug, it is not limited to, but the method immersing the biodegradable inverted-opal structure in a solution including the drug is exampled.

As for the method for burying the biodegradable inverted-opal structure having the pore holding the drug into a biomedical tissue, for example, the method employing a trocar, which is used in a laparoscopical operation, is exampled.

According to the method for using the biodegradable inverted-opal structure of the present invention, the holding-drug is released by biodegrading and/or responding to pH.

At first, the drug-release by the biodegradation will be explained.

In order to release the drug by the biodegradation, ion-exchange water, a buffer solution whose pH is regulated to acid or alkaline, and a solution including an enzyme at a proper concentration are used. Such solutions are used for assessing the biodegradability according to the hydrolysis reaction and for regulating the degradation reaction speed. Thus, the above-solutions enable to regulate the drug-release speed. In addition, considering the applications for the medical implant, it is desirable that the fully-degraded-time of the biodegradable inverted-opal structure is ranged from about a few weeks to 1 year.

Next, the drug-release by the pH response will be explained. The structure of the present invention includes a carboxyl group which is not used for the ester-bond therein, and further it has a large specific surface area, and therefore it shows a high mechanical response to pH change. For example, under high pH conditions, the proton is dissociated from the carboxyl group and electrostatic repulsion occurs between negative charges. This results in that the volume is expanded. On the other hand, under low pH conditions, the proton is added to the carboxyl group, and negative charges are neutralized. This results in that the volume is contracted. These mechanical properties may be repeatedly changed if its hydrolysis effect is ignored. Further, the drug may be intermittently released by the autonomous response to pH change.

The biodegradable inverted-opal structure of the present invention has biodegradability, and therefore it is gradually degraded by buffer solution, enzyme or the like. The pore diameter and three-dimensional-regularity of the pore are changed by the biodegradation and the mechanical response to pH change. By measuring them, the drug-release amount is detected. A reflection measurement device comprising a spectral apparatus, a light source and a detecting probe is enough to be used for the measurement. The reflection measurement device has a compact size, which is unlike X-ray CT and MRI, so that the actual time measurement is rapidly and easily carried out at bedside and the burden of patients is reduced.

In order to detect the change of the pore diameter, the visible and near-infrared light having a wavelength of 600 to 1100 nm, which has high penetration in a biomedical tissue, is preferably used as an incident light source. Specifically, the near-infrared light having a wavelength of 700 to 1000 nm, which is referred as “atmospheric window region”, is excellent in tissue penetration, and for example, the near-infrared light having a wavelength of 830 nm has a penetration depth of 1300 nm. According to the present invention, the pore diameter of the biodegradable inverted-opal structure may be easily regulated, and thus the light in desired regions may be selected.

The reflectance spectrum may be measured by a normal spectrophotometer, however, in order to observe its biodegradable process with the actual time on the moment, a reflection measurement system comprising a fiber-optic compact spectrophotometer, a light microscope and a CCD camera is preferably used. In the system, as for the incident light source, a white light source such as halogen light source and xenon light source, or a monochromatic light source such as solid-state laser and laser diode is used.

Next, a method for measuring the release amount of the drug held in the biodegradable inverted-opal structure of the present invention will be specifically explained.

The drug is released by the biodegradation and the response to pH as explained above. For example, when the drug is released by the biodegradation, the adhered or adsorbed drug is time-released during the process of the biodegradable inverted-opal structure being disrupted. Alternatively, the drug may be released by the volume swelling and contraction of the structure associated with pH change.

In order to measure the release-amount, a pseudo drug such as methylene blue absorbing visible light is used. The release-amount of the pseudo drug is measured by the absorbance of visible absorption spectrum, and the change of wavelength and strength of the reflected light resulting from the biodegradation is measured. By correlating between the two results, the release-amount is obtained.

The biodegradable inverted-opal structure of the present invention is further used as a separating membrane for biological material, a cell culture medium, a wound dressing (or artificial skin) or the like.

For the detail, when the structure is made as the separating membrane for biological material, the membrane may be used for separating biological materials such as protein and DNA by taking advantage of the porous structure having the size of several hundred nanometers. Further, condition of adhesion of materials to the pore inner wall may be confirmed according to the change of reflection property.

When the structure is used as the cell culture medium, cells may be grown and proliferated in the structure. In this case, the cell growth and proliferation conditions may be observed by referring to the change of reflection property associated with the biodegradation of the biodegradable inverted-opal structure.

The structure may be used as the wound dressing (or artificial skin). In this case, the porous structure, which the biodegradable inverted-opal structure has, allows gas or water to be exchanged therethrough. Further, the absorption condition in the biological body may be observed by referring to the change of reflection property.

Thus, the biodegradable inverted-opal structure of the present invention excels in the following respects compared to the conventional materials used in the body.

According to the invention disclosed in Patent document 1, a polymer gel is synthesized by using an organic solution and a polymerization initiator. On the other hand, the biodegradable inverted-opal structure of the present invention comprises a copolymer comprising polyhydric carboxylic acid, polyhydric alcohol, hydroxycarboxylic acid and lactone-group, and the organic solvent and the polymerization initiator are not necessary. Therefore, the structure excels in that the biological toxicity such as unreacted reagents and residues does not exist therein.

Compared to the structure comprising a composition of polylactic acid disclosed in Patent document 2, the biodegradable inverted-opal structure of the present invention excels in the responsiveness to the physicochemical environmental-changes, and capable of not only releasing drugs continuously by its natural decomposition but also releasing the drugs intermittently and rapidly based on the mechanical response to pH change in a biomedical tissue. Also, the present structure excels in compatibility with hydrophilic conditions such as biomedical tissue and ability for holding a drug having hydrophilic property. In addition, the biodegradable inverted-opal structure of the present invention is manufactured in a simple and easy way. On the other hand, the method for manufacturing the structure disclosed in Patent document 2 has a difficulty in regulating preparation conditions of the porous substrate which is used as the template. Further, the substrate is not suitable for a polymer having low fluidity and a polymer gel because the polymer or polymer gel may not permeate multiple pores. Further, according to the method for manufacturing the biodegradable inverted-opal structure of the present invention, the obtained structure has an internal space which is relatively big, and therefore enables to hold the desired amount of the drug.

In Patent document 3 discloses the medical implant comprising a biodegradable polymer. It is also described that the implant releases a drug continuously. Compared to the implant, the biodegradable inverted-opal structure of the present invention advantageously releases the drug intermittently especially when the drug has strong side-effects. In addition, the biodegradable inverted-opal structure of the present invention excels in that large equipments such as X-ray CT and MRI are not necessary in measuring its drug-release amount.

Compared to the two-dimensional mesh structure disclosed in Patent document 4, the biodegradable inverted-opal structure of the present invention has a three-dimensionally-periodic-ordered-pore, so that it shows the selective light reflection property and high mechanical responsiveness. Thus, in order to measure the residual volume during the biodegradation, the structure disclosed in Patent document 4 requires large equipments such as X-ray CT and MRI after the structure is buried in a biomedical tissue which are physically-taxing to the treated patients. On the other hand, the biodegradable inverted-opal structure of the present invention is capable of selectively reflecting the near-infrared light having high tissue penetration due to the inverted-opal formation. Therefore, the residue amount is high-sensitively and noninvasively measured during the biodegradation by an optical means in a simple and easy way. For the measurement, a compact size spectrometer may be used, and the measurement may be carried out at bedside and therefore the burden of patients is reduced.

In the composition comprising a biodegradable polymer disclosed in Patent document 5, an organic solvent is used for the synthesis. On the other hand, the biodegradable inverted-opal structure of the present invention may be a copolymer of polyhydric carboxylic acid, polyhydric alcohol, hydroxycarboxylic acid and lactone-group, as well as may be a non-straight-chain-polymer having a branched-chain structure (polymer gel). Therefore, water may be used as a solvent for the synthesis. Thus, the troublesome procedures such as removing an organic solvent completely are unnecessary. In addition, the structure of the present invention has no need to use a polymeric initiator or a catalyst during the thermal polymerization, and therefore the procedure for their removal is unnecessary. Further, the structure of the present invention has the inverted-opal formation and shows the reflection property and high mechanical responsiveness. However, the biodegradable polymer disclosed in Patent document 5 is a non-porous structure, and therefore such properties are not expected.

The inverted-opal structure comprising a composition including a sulfide series compound such as episulfide compound as an essential component is disclosed in Patent document 6. The structure, which is aimed at being applied to optical devices such as optical filter, optical waveguide and laser cavity, consists of a compound having high refractive index and therefore it is inappropriate for medical materials. Thus, the structure does not have biodegradability and biocompatibility (ex. non-stimulus property, low drug toxicity caused by degraded products) which are required for use in biomedical tissue. On the other hand, the biodegradable inverted-opal structure of the present invention aimed at being provided as an implant material used in a biomedical tissue. Specifically, as a component, the low molecular compound having low drug toxicity is selected, and the polymer of the low molecular compound is designed to be degraded relatively easily by the hydrolysis reaction under body environment. Further, the biodegradable inverted-opal structure of the present invention is a flexible gelled compound, so that it has an advantage that the mechanical stimulus to the biomedical tissue is low.

In Non Patent document 1, although it is disclosed that a polyester gel comprising an aliphatic alcohol and an aliphatic carboxylic acid has biodegradability and pH responsiveness, only non-porous body which does not have an inverted-opal structure is mentioned. The structure of the present invention has a property of selectively reflecting light ranged from visible to near-infrared regions when the pore size is about several hundred nanometers. This property is commonly known in structures whose refractive index is changed periodically at about light wavelength period. On the other hand, the structure disclosed in Non Patent document 1 consists of non-porous body having a uniform refraction index, and does not show the reflecting property as shown in the present invention. Thus, the biodegradable inverted-opal structure of the present invention has a large specific surface area, and therefore excels in the mechanical response speed (ex. swelling and contraction) against external stimulus such as pH. However, the non-porous body disclosed in Non Patent document 1 does not have such a property.

### EXAMPLES

The present invention is explained by presenting examples below in order to make the effect clear, but the invention is not limited to the following examples.

### (The Synthesis of the Biodegradable Inverted-Opal Structure: 1)

A suspension including silica particles having average diameter of 300 nm (Polysciences, Inc.) was delivered by drops on a glass substrate by using a pasteur pipette. After placing in a darkroom at normal temperature and humidity, a colloidal crystal thin-film was obtained.

As a material of the biodegradable inverted-opal structure, citric acid (L.D.₅₀ (oral mouse)=5,040 mg/kg) (made by Wako Pure Chemical Industries, Ltd.), pentaerythritol (25,500 mg/kg), 1,5-pentanediol (25,500 mg/kg), which are known as its low toxicity, were used. Pentaerythritol 0.0681 g (0.5 mmol), 1,5-pentanediol 0.52 g (5 mmol) and citric acid 1.153 g (6 mmol) were dissolved in ion-exchange water and fully dissolved at room temperature. The obtained mixed solution was delivered by drops into the above-prepared colloidal crystal thin-film by using a pasteur pipette, the thin-film was immersed in the mixed solution, and excess solution was removed with a Kimwipe tissue. Subsequently, the glass substrate including the thin-film was transferred to a 100 ml pressure bottle, ion-exchange water was added thereto, and the thermal polymerization was carried out by being heated at 127° C. for 24 hours in an oven. After this operation, a polyester thin-film including a colloidal crystal therein, which is a composition of a colloidal crystal coated with aliphatic polyester of the present invention, was obtained.

In an etching solution including Dimethylsulfoxide, 42% hydrofluoric acid ammonium aqueous solution and ethanol (made by Wako Pure Chemical Industries, Ltd.), the above-obtained polyester thin-film was immersed together with the glass substrate, and maintained for 5 to 48 hours in order for the silica particle to be eluted. Also, after the treatment, the polyester thin-film was separated from the glass substrate, and the biodegradable inverted-opal structure of the present invention was obtained. After the structure was washed with ion-exchange water, it was preserved in an ethanol preservative solution.

### (Electron Microscope Observation)

Using the polyester thin-film obtained from the above-operation, the scanning electron microscope observation was carried out (measurement device: made by Hitachi High-Technologies Corporation, Ultrahigh resolution field emission scanning electron microscope S-4800). The structure just after being vacuum-freeze-dried, which had been picked up from the ethanol preservative solution and then washed with ion-exchange water, was used as a sample.

In an electron micrograph (shown in FIG. 2), it was confirmed that the biodegradable inverted-opal structure of the present invention undergoing the etching for 5 hours had a periodic mesh structure. In the colloidal crystal used for the template, a crystal grew in (111) orientation of the vertical direction in the glass substrate. The micrograph shows a hexagonal structure reflecting the crystal. Further, the residues shown in the pores were silica particles which were used as the template, and it was considered that the etching was insufficient.

An electron micrograph of the biodegradable inverted-opal structure of the present invention undergoing the etching for 30 hours (shown in FIG. 3) shows the structure has regularity. However, it was also confirmed that the pore diameter was reduced resulting from removal of silica particles used for the template from the structure, and no-existence of solvent in the sample. In addition, the micrograph does not show the residues of the silica particles in the pores. In the structure undergoing the etching for 48 hours, the silica particles were completely removed. This was confirmed with a composition analysis by using an energy dispersive X-ray analyzer (made by Horiba, Ltd. EMAX-ENERGY).

### (Infrared Absorption Spectrum Measurement)

The result of the infrared absorption spectrum measurement (measurement device: made by JASCO Corporation FT/IR-470) is shown in FIG. 4. The biodegradable inverted-opal structure of the present invention obtained from the above-mentioned method (The synthesis of the biodegradable inverted-opal structure: 1), which had undergone the etching for 48 hours, was picked up from the ethanol preservative solution, washed with ion-exchange water, and vacuum freeze-dried for 24 hours. The resultant was used as a sample. The infrared absorption spectrum was shown in FIG. 4-2. The FIG. 4-1 and 4-3 shows the infrared absorption spectrum of the silica particle and the mixture of monomers, respectively.

In the spectrum of the silica particle (FIG. 4-1), extremely strong absorption derived from the stretching vibration of Si—O—Si bond was shown around 1000-1300 cm⁻¹. However, such an absorption was not shown at all in the spectrum of the biodegradable inverted-opal structure undergoing the etching for 48 hours (FIG. 4-2). This shows that the silica particle was completely removed by the etching. In addition, in the spectrum of the mixture of monomers (FIG. 4-3), strong absorption derived from the stretching vibration of C═O bond and C—O bond was shown around 1740 cm⁻¹ and 1220 cm⁻¹, respectively. On the other hand, in the spectrum of the biodegradable inverted-opal structure, the absorption around 1740 cm⁻¹ was weak and broad-ranging, and the absorption around 1220 cm⁻¹ was not observed clearly. The measured results suggested that ester-bond was formed between hydroxyl group and carboxyl group which are comprised in the monomers, a mesh gel was formed, and therefore C═O bond and C—O bond were under several different chemical conditions.

### (Raman Spectrum Measurement)

The result of the raman spectrum measurement of a polyester having the same composition which was synthesized under the same conditions as the biodegradable inverted-opal structure (measurement device: made by Thermo Electron, FT-IR-Raman Spectrometer Nexus 870) is shown in FIG. 5. In the spectrum, the peaks shown in 1305 cm⁻¹ and 1733 cm⁻¹ are derived from the characteristic oscillation of C—O—C bond and C═O bond, respectively, and the results show that the ester-bond was formed by the thermal polycondensation during the above-mentioned synthesis method.

### (Reflectance Spectrum Measurement)

The change of reflection property of the biodegradable inverted-opal structure associated with pH responsiveness was examined. For the measurement, the biodegradable inverted-opal structure undergoing the etching for 48 hours was picked up from the ethanol preservative solution, and washed with ion-exchange water. Subsequently, the glass substrate including the structure was transferred to a styrene case and immersed in an aqueous sodium hydroxide (pH=1.5). In order to fix the polyester thin-film, a cover glass was used. The case including the polyester thin-film was placed on the stage of a light microscope (made by Nikon Corporation, industrial light microscope ECLIPSE LV100D), and the change of reflectance spectrum of the sample was measured (measurement device: made by Ocean Optics, Inc., reflection measurement high resolution fiber multi-channel spectroscopic system).

The change in reflectance spectrum with time was shown in FIG. 6. In FIG. 6, the additional characters show the time change (1: 0 minute, 2: 87 minutes, 3: 130 minutes, 4: 201 minutes, 5: 440 minutes, 6: 1046 minutes, 7: 3320 minutes) after being immersed in the aqueous sodium hydroxide. This shows that, before being immersed, the sample had a maximum reflected wavelength at 679 nm, and after being immersed, the peak position shifted to the long-wavelength side with time and ultimately reached to the near-infrared region (797 nm). This is because the pore diameter was enlarged resulting from the electrostatic repulsion caused by the proton dissociation from the carboxyl group of polyester and the swelling associated with the hydrophilic improvement. In addition, the reflected intensity tended to be lower. This results from the reduced-difference between the refraction index of the swollen polyester and the refraction index of the solution existing in the pores. The maximum reflected intensity and the maximum reflected wavelength with time are shown in FIG. 7 and FIG. 8, respectively.

The changes of reflection property of the biodegradable inverted-opal structures which occur before and after the hydrolysis are shown in FIG. 9. In FIG. 9, the biodegradable inverted-opal structures before and after being immersed in a pH buffer solution are identified by each additional character. The character 1 means before being immersed and the character 2 means after 45 hours being immersed. The above sample was immersed in an aqueous hydrochloric acid regulated to pH 3.0 for about 3 days and then washed with ion-exchange water. The resultant was used as a sample for the measurement. As for a buffer solution, carbonate pH standard solution second class (pH 10.01, made by Wako Pure Chemical Industries, Ltd.) was used. It was confirmed that the polyester was completely hydrolyzed in the buffer solution, and the reflection derived from the inverted-opal structure was disappeared.

The pH dependency of the reflectance spectrum is shown in FIG. 10. In FIG. 10, the additional characters show the orders of immersing the biodegradable inverted-opal structure in the solution, and 1 and 3 are pH=3, and 2 and 4 are pH=11. The measurement was carried out by immersing the sample to a hydrochloric acid solution (pH=3) and an aqueous sodium hydroxide (pH=11) alternately. In pH=3, the reflection peak was shown in shorter wavelength side compared to the above-reflected wavelength. The reason is considered that, in low pH, the effect of the electrostatic repulsion between carboxyl groups is decreased by the proton addition to the carboxyl group of polyester, and therefore the pore diameter was reduced. On the other hand, in pH=11, the reflection peak was shown in the long wavelength side. The reason is considered that proton was dissociated from the carboxyl group, carboxyl groups were electrostatically repulsed each other, and therefore the pore diameter was enlarged. In FIG. 10, the repeatability of the peak shift of the maximum reflected wavelength associated with pH responsiveness was confirmed.

### (Optical Microscope Observation)

The structural color of the biodegradable inverted-opal structure during the hydrolysis process was examined with a microscope digital system (made by Shimadzu rika corporation, Moticam2000). For the hydrolysis of the sample, ion-exchange water (pH 6-7) was used. The observation photographs of the sample just after being immersed in ion-exchange water and the sample being immersed in ion-exchange water for 284 hours are shown in FIG. 11 and FIG. 12, respectively.

Further, observation photographs of a non-inverted-opal structure disclosed in Non Patent document 1 are shown in FIG. 13 and FIG. 14. The biodegradable inverted-opal structure of the present invention (FIG. 11) shows the selective light reflection (i.e., structure color) derived from the inverted-opal formation. On the other hand, non-inverted-opal structures shown in FIG. 13 and FIG. 14 are non-porous bodies which do not have the inverted-opal formation, and therefore they are transparent and colorless.

### (Refractive Index Measurement)

As for the non-porous polyester having the same composition as the biodegradable inverted-opal structure of the present invention, its refractive index was measured (measurement device: made by ATAGO corporation, Abbe refractometer NAR-1T), and the result was n_{D}=1.49. By using the refractive index, the average refractive index of the biodegradable inverted-opal structure of the present invention was calculated with the following formula.

na²=Σnᵢ²Vᵢ (Formula 1)

Here, nₐ means an average refractive index between a polyester which is a component of the structure and a component of the interior pore, nᵢ means a refractive index of each component, and Vᵢ means a volume fraction of each component. Because the periodical sequence of the pore is a face-centered cubic structure, the volume fraction of the pore is 0.74 and the volume fraction of the polyester is 0.26. When water (n_{D}=1.33) is inside pore, the pore diameter is assumed to be same as the particle diameter (300 nm) of a colloidal particle which is used as the template, the average refractive index is estimated as nₐ=1.37. By using the average refractive index, the diffracted wavelength of the reflected light was calculated with the following formula, and the value was 673 nm.

λ=1.633(d/m)(na²−sin²θ)^{1/2} (Formula 2)

Here, d: shows a pore diameter and m: shows Bragg constant (m=1). The diffracted wavelength obtained from the reflection measurement was about 670 nm, which showed that the calculated average refractive index (nₐ=1.37) was reasonable. This supports that the structure of the present invention has an inverted-opal formation.

### (The Synthesis of the Biodegradable Inverted-Opal Structure: 2)

In order to synthesize the biodegradable inverted-opal structure, a polylactic acid was used. The polylactic acid was used for bone-bonding material, suture thread, drug carrier, or the like, and it was known to have biodegradability and biocompatibility.

A 30% wt acetone solution of DL-polylactic acid (Taki Chemical Co., Ltd.) was delivered by drops into a colloidal crystal thin-film by using a pasteur pipette, and the thin-film was immersed in the solution. Subsequently, after placing the solution at normal temperature and humidity for 1 day, the colloidal crystal coated with the DL-polylactic acid was obtained. The colloidal crystal film was manufactured by using a suspension including silica particles having the average diameter of 400 nm (made by Polysciences, Inc).

The above-thin film was immersed in a 2.3% wt aqueous hydrofluoric acid (made by Wako Pure Chemical Industries, Ltd.), placed in a dark cold place for 48 hours, and the silica sol was eluted. Subsequently, the thin-film was washed with ion-exchange water, immersed in ion-exchange water, and preserved in a dark cold place.

### (Electron Microscope Observation)

The electron micrograph (FIG. 15) shows a biodegradable inverted-opal structure comprising a polylactic acid manufactured by using a silica particle having the diameter of 400 nm. The porous structure reflecting the three-dimensionally periodic structure of the colloidal crystal which was used as the template was confirmed.

The structural change caused by the biodegradation of the above structure was examined. The electron micrograph (FIG. 16) shows the sample after being buried in a mouse subcutaneous tissue for one week, and it shows that the pore periodicity and the pore size uniformity in the inverted-opal structure are both lost by the biodegradation.

In addition, any marked inflammation was not confirmed in the mouse subcutaneous tissue where the structure was buried, and the mouse body weight did not particularly show a decreasing trend, and therefore it was suggested that the biodegradable inverted-opal structure of the present invention has biocompatibility.

### (Reflectance Spectrum Measurement)

The reflection property of the biodegradable inverted-opal structure of the present invention is shown in FIG. 17-1. This shows that the reflection peak can be regulated to around 860 nm by using a silica sol having a diameter of 400 nm during the synthesis.

The FIG. 17-2 shows a reflectance spectrum obtained when a mouse subcutaneous tissue was placed on the biodegradable inverted-opal structure. In the measurement, a halogen lamp was used as a light source. Compared to the reflection peak of FIG. 17-1, the reflected intensity was low. However, a clear reflection peak was observed. This peak results from that the incident light and reflected light are not completely absorbed by the skin tissue and pass through the skin tissue, because the reflection peak of the above-biodegradable inverted-opal structure is positioned in the near-infrared region.

### BRIEF DESCRIPTION OF THE DRAWINGS

FIG. 1 shows processes of manufacturing a biodegradable inverted-opal structure from a colloidal crystal by the manufacturing method of a biodegradable inverted-opal structure according to the present invention. (1) shows the colloidal crystal, (2) shows the composition of the colloidal crystal coated with an aliphatic polyester, (3) shows the biodegradable inverted-opal structure.

FIG. 2 shows an electron micrograph of one example of a structure after undergoing an etching to a biodegradable inverted-opal structure of the present invention for 5 hours.

FIG. 3 shows an electron micrograph of one example of a structure after undergoing an etching to a biodegradable inverted-opal structure of the present invention for 30 hours.

FIG. 4 shows a graph of one example about identification results of a biodegradable inverted-opal structure of the present invention. 1, 2 and 3 show the infrared absorption spectrums of a silica particle, a biodegradable inverted-opal structure of the present invention and a mixture of monomers, respectively.

FIG. 5 shows a graph of one example about a raman spectrum of a polyester being synthesized under the same conditions and having the same composition as a biodegradable inverted-opal structure of the present invention.

FIG. 6 shows a graph of one example about changes in reflectance spectrums with time during a process of pH response of a biodegradable inverted-opal structure of the present invention. The additional characters show the elapsed time (1: 0 minute, 2: 87 minutes, 3: 130 minutes, 4: 201 minutes, 5: 440 minutes, 6: 1046 minutes, 7: 3320 minutes).

FIG. 7 shows a graph of one example about a change in the maximum reflected intensity with time during a process of pH response of a biodegradable inverted-opal structure of the present invention.

FIG. 8 shows a graph of one example about a change in the maximum reflected wavelength with time during a process of pH response of a biodegradable inverted-opal structure of the present invention.

FIG. 9 shows a graph of one example about changes of reflectance spectrums of biodegradable inverted-opal structures of the present invention before and after the hydrolysis. The additional characters show the elapsed time (1: 0 hour, 2: 48 hours).

FIG. 10 shows a graph of one example about changes of reflectance spectrums associated with pH change of a biodegradable inverted-opal structure of the present invention. The additional characters show the orders of immersing a sample in a solution, and 1 and 3 are pH=3, and 2 and 4 are pH=11.

FIG. 11 shows an optical micrograph of one example about a structural color of a biodegradable inverted-opal structure of the present invention.

FIG. 12 shows an optical micrograph of one example about a structural color of a biodegradable inverted-opal structure shown after the hydrolysis of the present invention.

FIG. 13 shows a photograph of one example of a non-inverted-opal structure (non-porous body).

FIG. 14 shows an electron micrograph of one example of a non-inverted-opal structure (non-porous body).

FIG. 15 shows an electron micrograph of one example of a structure of a biodegradable inverted-opal structure of the present invention.

FIG. 16 shows an electron micrograph of one example of a structure during its biodegrading process of a biodegradable inverted-opal structure of the present invention.

FIG. 17 shows one example of reflection properties of a biodegradable inverted-opal structure of the present invention. (In the FIG. 1 is a reflection property obtained when nothing was placed on a sample, and 2 is a reflection property obtained when a mouse subcutaneous tissue was placed on a sample).

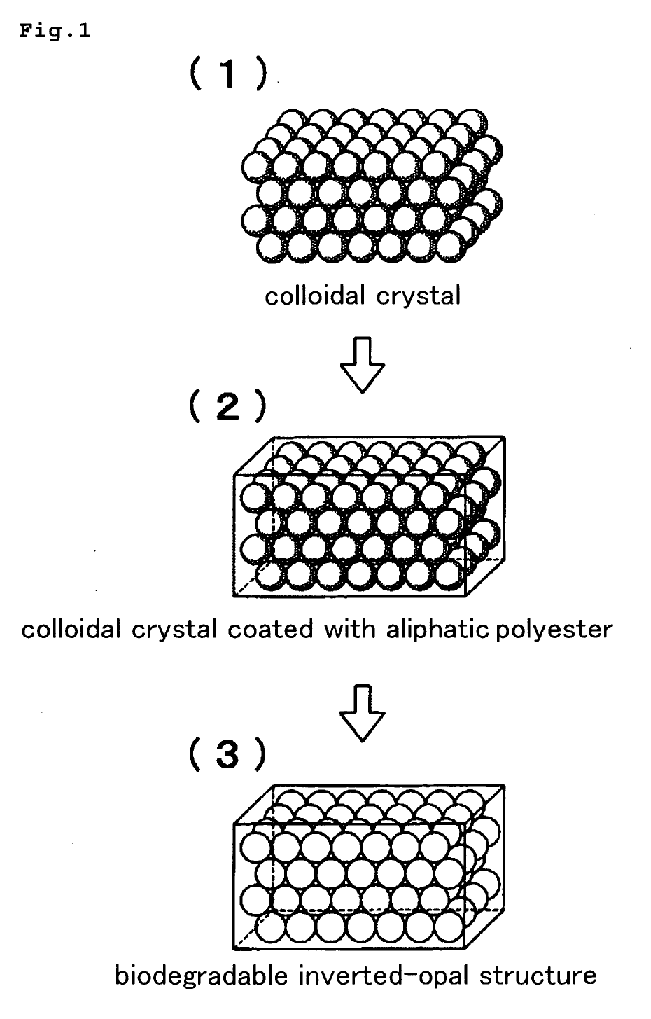

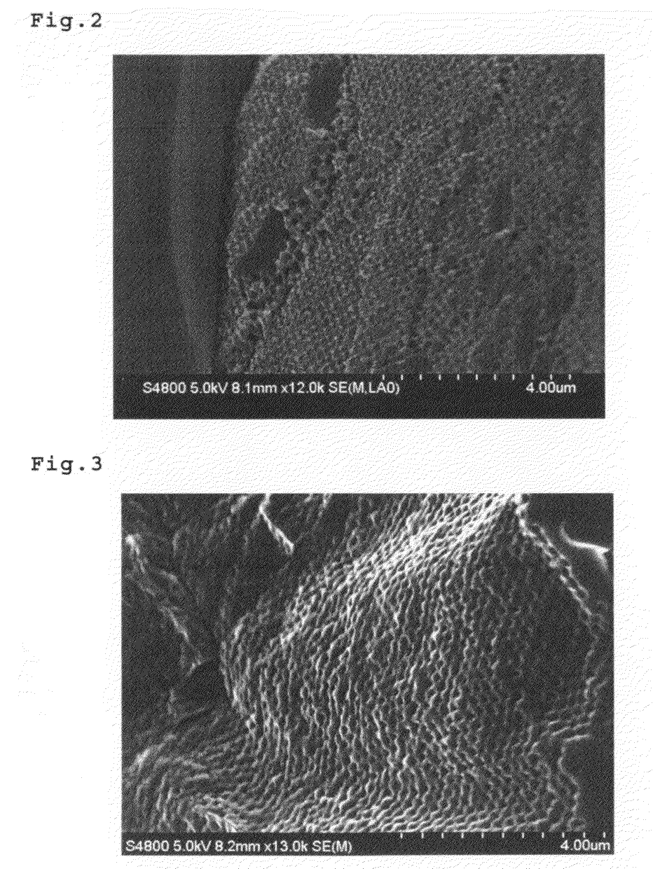

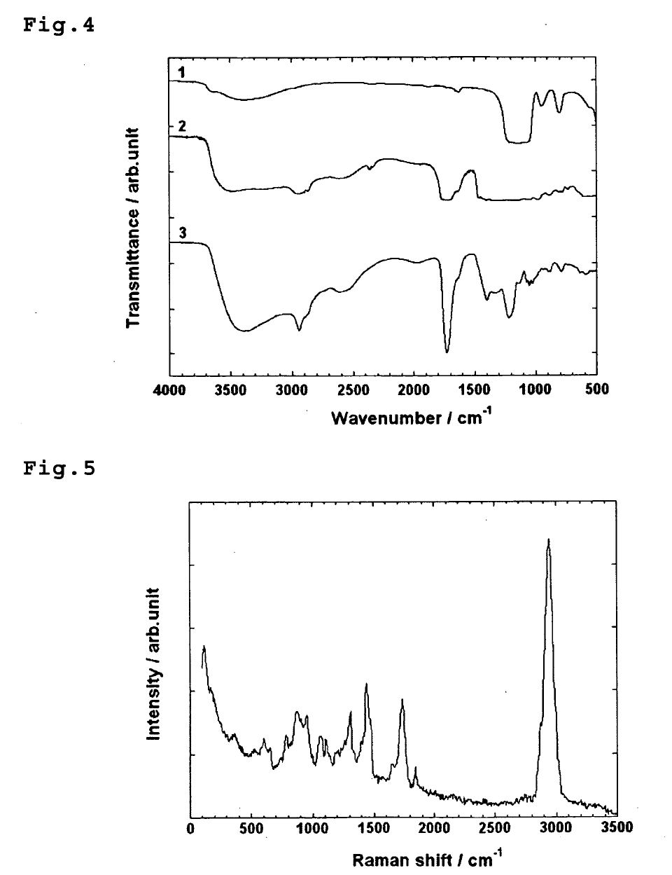

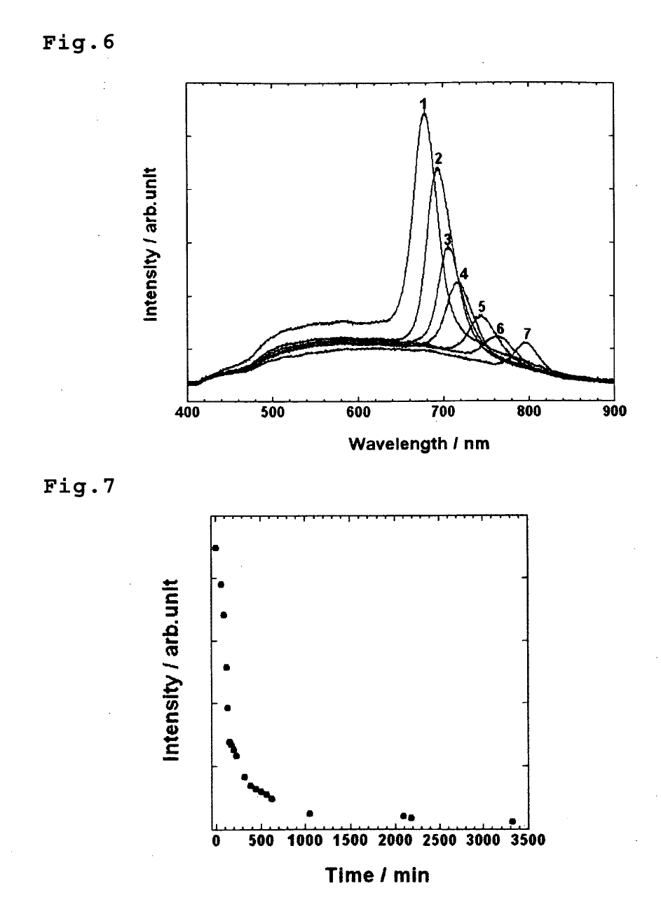

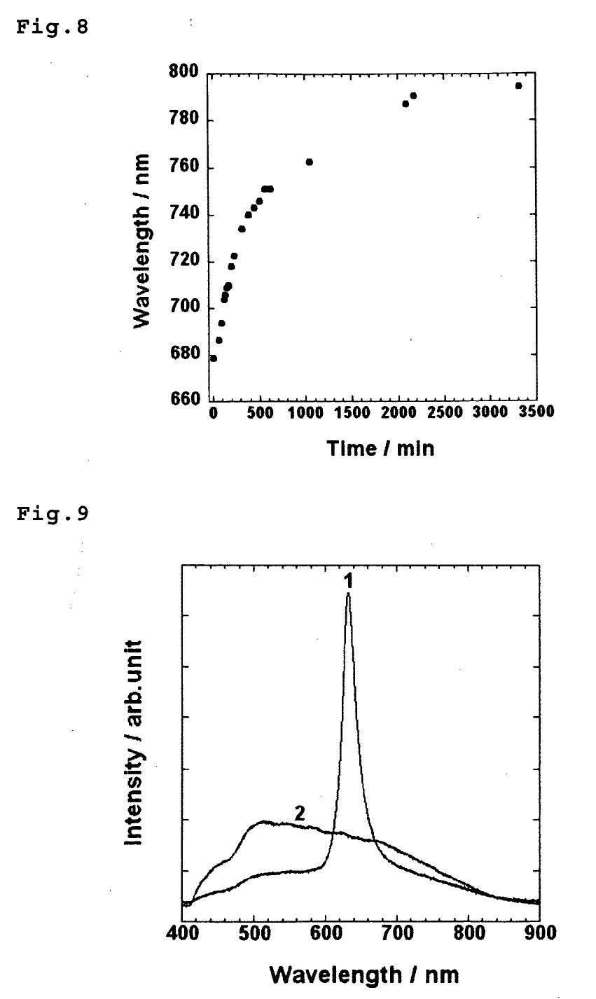

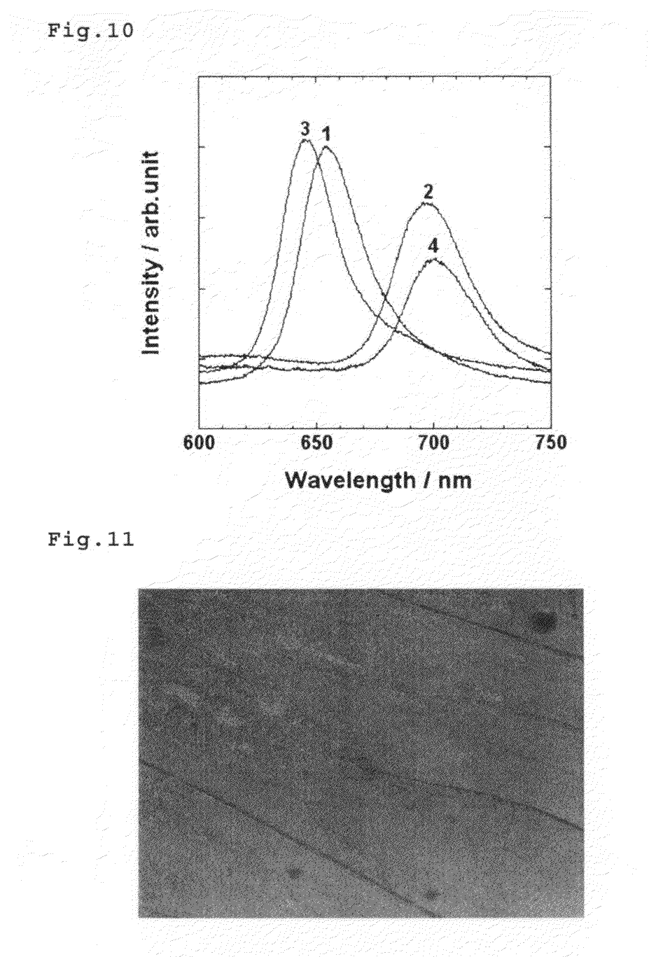

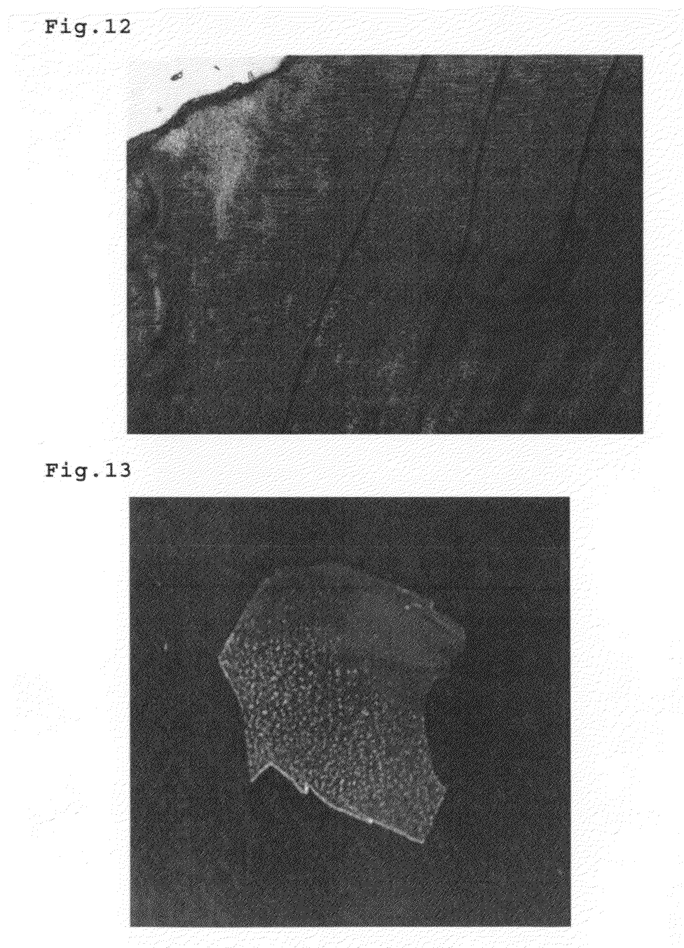

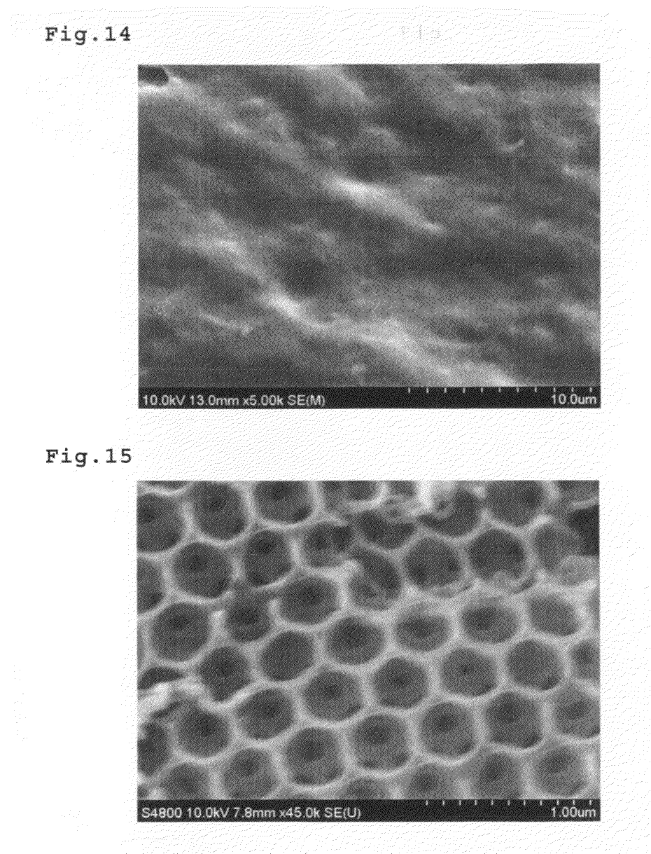

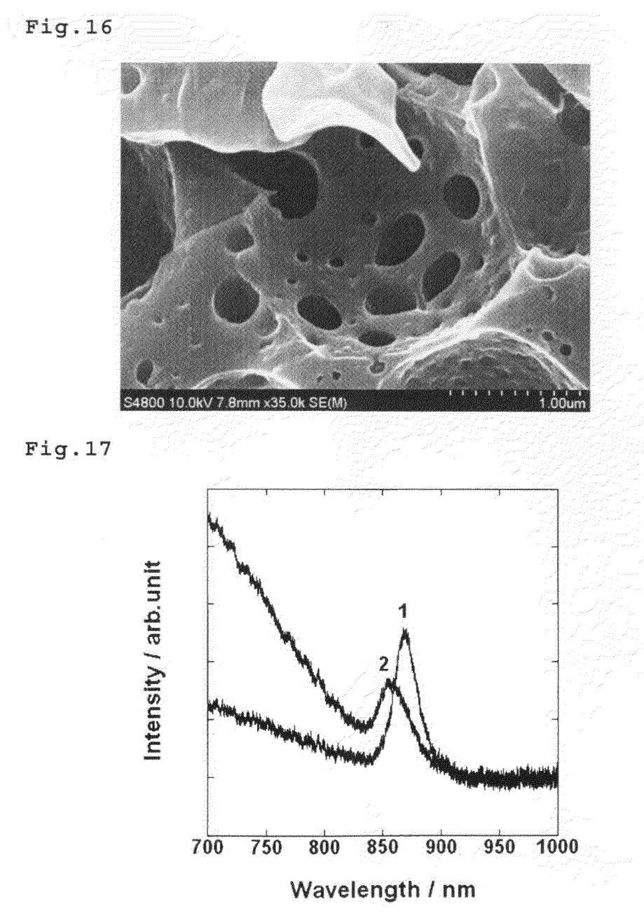

1. A biodegradable inverted-opal structure comprising an aliphatic polyester.

2. The biodegradable inverted-opal structure according to claim 1,
wherein said inverted-opal structure has three-dimensionally-ordered-pores, and said pore selectively reflects light in visible and near-infrared regions.

3. The biodegradable inverted-opal structure according to claim 2,
wherein said light in visible and near-infrared regions has a wavelength of 600 to 1100 nm.

4. The biodegradable inverted-opal structure according to claim 2,
wherein said pore has a diameter of 10 to 1000 nm.

5. The biodegradable inverted-opal structure according to claim 1,
wherein said aliphatic polyester is formed by ester-bonding between monomers selected from the group consisting of polyhydric carboxylic acid, polyhydric alcohol, hydroxycarboxylic acid and lactone-group.

6. The biodegradable inverted-opal structure according to claim 2,
wherein said aliphatic polyester is formed by ester-bonding between monomers selected from the group consisting of polyhydric carboxylic acid, polyhydric alcohol, hydroxycarboxylic acid and lactone-group.

7. The biodegradable inverted-opal structure according to claim 6,
wherein said aliphatic polyester comprises said monomers in a composition rate ranged from 0.001 to 1000% by weight respectively.

8. The biodegradable inverted-opal structure according to claim 1,
wherein said aliphatic polyester is a polylactic acid.

9. The biodegradable inverted-opal structure according to claim 2,
wherein said aliphatic polyester is a polylactic acid.

10. The biodegradable inverted-opal structure according to claim 1,
wherein said inverted-opal structure has a pH responsiveness.

11. The biodegradable inverted-opal structure according to claims 2,
wherein said inverted-opal structure has a pH responsiveness.

12. A medical implant comprising the biodegradable inverted-opal structure according to claim 1.

13. A medical implant comprising the biodegradable inverted-opal structure according to claim 2.

14. A composition of a colloidal crystal coated with an aliphatic polyester manufactured by a method comprising the steps of:
(1) producing a colloidal crystal from a silica particle or a polystyrene particle;
(2) immersing the colloidal crystal in a solution including a monomer from which the aliphatic polyester is formed; and
(3) thermally-polymerizing the monomer under a pressurized condition in order to obtain a composition of the colloidal crystal coated with the aliphatic polyester.

15. The composition of the colloidal crystal coated with the aliphatic polyester according to claim 14,
wherein said silica particle or polystyrene particle has a weight fraction of 0.01-90% by weight.

16. A method for manufacturing a biodegradable inverted-opal structure, comprising the steps of:
(1) producing a colloidal crystal from a silica particle or a polystyrene particle;
(2) immersing the colloidal crystal in a solution including a monomer from which the aliphatic polyester is formed;
(3) thermally-polymerizing the monomer under a pressurized condition in order to obtain a composition of the colloidal crystal coated with the aliphatic polyester; and
(4) removing the silica particle from said composition by etching, or removing the polystyrene particle from said composition by eluting the polystyrene particle with an organic solvent in order to obtain the biodegradable inverted-opal structure.

17. A method for using a biodegradable inverted-opal structure comprising an aliphatic polyester, comprising a step of releasing a drug from the biodegradable inverted-opal structure in vivo by biodegrading and/or responding to pH after holding said drug in the biodegradable inverted-opal structure.

18. A method for measuring a drug-release amount in vivo from a biodegradable inverted-opal structure comprising an aliphatic polyester, comprising the steps of:
(a) releasing a drug from the biodegradable inverted-opal structure in vivo by biodegrading and/or responding to pH after holding said drug in the biodegradable inverted-opal structure; and
(b) entering light in visible or near-infrared region into said biodegradable inverted-opal structure, and measuring the change of wavelength and strength of the reflected light.

19. A method for measuring a drug-release amount in vivo from a biodegradable inverted-opal structure comprising an aliphatic polyester according to claim 18, further comprising the steps of:
(i) releasing a pseudo drug from the biodegradable inverted-opal structure in vivo by biodegrading and/or responding to pH after holding said drug in the biodegradable inverted-opal structure; and
(ii) entering light in visible or near-infrared region into said biodegradable inverted-opal structure, measuring (A) change of wavelength and strength of the reflected light, measuring (B) drug-release amount of said pseudo drug by a quantitative analysis of visible absorption spectrum, and correlating (A) with (B).

20. A method for enlarging a pore diameter of a biodegradable inverted-opal structure comprising an aliphatic polyester, comprising a step of hydrolyzing the inner wall of the pore of the biodegradable inverted-opal structure.
